(19) United States
(12) Patent Application Publication
(10) Pub. No.: US 2024/0207223 A1
(43) Pub. Date: Jun. 27, 2024

(54) VITAMIN C PREPARATION

(71) Applicant: PEDRO P. PEREZ, Miami Lakes, FL (US)

(72) Inventor: PEDRO P. PEREZ, Miami Lakes, FL (US)

(21) Appl. No.: 18/108,509

(22) Filed: Feb. 10, 2023

Related U.S. Application Data
(63) Continuation of application No. 18/079,344, filed on Dec. 12, 2022.

(51) Int. Cl. A61K 31/375; A61K 31/352; A61K 47/10; A61K 47/12

(52) U.S. Cl. CPC A61K 31/375; A61K 31/352; A61K 47/10; A61K 47/12

(57) ABSTRACT

The present invention relates to vitamin C preparations which enhance absorption of vitamin C into cells and prolong the retention of vitamin C within the blood plasma and tissue of mammals, such as humans. The vitamin C preparations of the present invention include lipophilic molecules which improve the absorption of vitamin C resulting in higher plasma and cellular levels.

### CLAIM OF PRIORITY

This is a continuing-in-part patent application of previously filed, now pending application having Ser. No. 18/079,344, filed on Dec. 12, 2022, which is incorporated herein by reference.

### BACKGROUND OF THE INVENTION

### Field of the Invention

The present invention relates to vitamin C preparations having enhanced bioavailability.

### Description of the Related Art

The term “vitamin C,” unless otherwise stated, refers to ascorbic acid and pharmaceutically acceptable salts thereof, including, but not limited to, mineral salts of ascorbic acid, effervescent vitamin C (e.g., a combination of ascorbic acid, citric acid and sodium bicarbonate), chelates of ascorbic acid, microencapsulated of vitamin C, liposomal of vitamin C, and alkaline salts of ascorbic acid, as would be understood by those skilled in the art.

According to the National Institute of Health and the Food and Nutritional Board of the National Academy of Science, vitamin C is an essential nutrient involved in many biological functions. Vitamin C can only be acquired through diet (i.e., food or nutritional supplement). Vitamin C has been implicated as an important dietary component as it is required for physiological and metabolic activities including the development of healthy neurons (Zhou et al., 2003; Weeks & Perez, 2007), prevention of neurodegenerative diseases (Boothby & Doering, 2005; Landmark 2006), wound healing (Kaplan et al., 2004; Marionnet C et al., 2006; Weeks & Perez, 2007), maintenance of a healthy immune system (Fay et al., 1994; Lehr et al., 1994; Weeks & Perez, 2007), excellent antioxidant and free radical scavenging capabilities, superior uptake, bioavailability and cell retention (Fay et al., 1994; Lehr et al., 1994; Boothby & Doering, 2005; Landmark 2006, Weeks & Perez, 2007), reduction of plasma levels of inflammatory and oxidative stress markers, reduces plasma levels of C-reactive protein and oxidized LDL, more rapidly absorbed and leads to higher serum vitamin C levels (Fay et al., 1994; Lehr et al., 1994; Zhou et al., 2003; Boothby & Doering, 2005; Landmark 2006, Weeks & Perez, 2007; Pancorbo & Vazquez & Fletcher, 2008).

Furthermore, vitamin C contributes to the health of the cardiovascular, immunological and nervous systems, and also supports healthy bone, lung, skin, and wound-healing activities. The mechanisms through which vitamin C works generally pertain to use as an enzyme cofactor or directly as an antioxidant. While extracellular vitamin C is important in free-radical scavenging, particularly with LDL metabolism and lung protection, most vitamin C antioxidant activity and enzyme support is intracellular. Therefore, the delivery of vitamin C across the cell membrane and into the cell compartments directly impacts how effective the vitamin C is and whether the vitamin C can arrive at the needed site for healthy function.

Given the importance of vitamin C, the bioavailability of vitamin C has been the focus of intense research. An improvement in absorption and retention of vitamin C in blood plasma or tissue would increase the beneficial effects of vitamin C. Thus, there is a continuing need for vitamin C preparations having enhanced bioavailability.

### SUMMARY OF THE INVENTION

In view of the continuing need for vitamin C preparations having enhanced bioavailability, the present invention relates to a vitamin C preparation which enhances absorption of vitamin C into cells and prolongs the retention of vitamin C within the blood plasma and tissue of mammals, such as humans. Furthermore, the inclusion of a lipophilic molecule component improves the absorption of vitamin C, resulting in higher plasma and cellular levels. Thus, the present invention advantageously provides for a means to enhance the bioavailability of vitamin C, an essential nutrient involved in many biological functions.

In advance of the foregoing, the term “about” can be defined as what one skilled in the art would understand “about” to mean, and the term “about” includes a 5% tolerance on both lower and upper bounds, if applicable.

In more specific terms, the vitamin C preparation-preferably formulated as an oral dosage form, such as a tablet, capsule, softgel, or gummy-is comprised of a homogenous mixture which is further comprised of a vitamin C component and a lipophilic molecule component. The vitamin C component of the homogenous mixture comprises at least about 90% by weight of the total weight of the preparation. In one embodiment of the present invention, the vitamin C component comprises about 0.01 g to 2.0 g of vitamin C. The lipophilic molecule component, on the other hand, comprises at least about 0.1% by weight of the total weight of the preparation. The lipophilic molecule component further comprises at least one saturated straight C₁₆-C₃₄ fatty alcohol and at least one unsaturated ω-9 C₁₈-C₂₄ fatty acid. By way of non-limiting example, the lipophilic molecule component may further comprise a combination of at least one of the following: (i) at least one saturated straight C₁₄-C₂₄ fatty acid; (ii) at least one unsaturated ω-3 C₁₆-C₂₄ fatty acid; (iii) at least one unsaturated ω-6 Cis-C₂₂ fatty acid; and (iv) at least one unsaturated ω-7 C₁₆-C₂₀ fatty acid. According to one embodiment of the present invention, the homogenous mixture comprises a weight ratio of vitamin C to lipophilic molecules ranging from about 1000:1 to about 10:1. According to another embodiment, the weight ratio ranges from about 100:1 to about 8:1.

In one embodiment of the present invention, the homogenous mixture may comprise a vitamin C component, a lipophilic molecule component, and a bioflavonoid component. In such an embodiment, the bioflavonoid component comprises at least about 0.1% by weight of the total weight of the preparation. By way of non-limiting example, the homogenous mixture may comprise at least about 90% by weight of the vitamin C component, at least about 0.1% to 5% by weight of the lipophilic molecule component, and at least about 0.1% to 5% by weight of the bioflavonoid component. By way of another non-limiting example, the homogenous mixture may comprise at least about 90% by weight of the vitamin C component, at least about 0.1% to 9% by weight of the lipophilic molecule component, and at least about 0.1% to 5% by weight of the bioflavonoid component.

In another embodiment of the present invention, the homogenous mixture may comprise at least about 0.1% by weight of at least one saturated straight C₁₆-C₃₄ fatty alcohol and at least about 0.01% by weight of one of at least one unsaturated ω-9 C₁₈-C₂₄ fatty acid, based upon 100% total weight of the preparation. By way of non-limiting example, the homogenous mixture may further comprise a combination of at least one of the following: (i) at least about 0.01% by weight of at least one saturated straight C₁₄-C₂₄ fatty acid; (ii) at least about 0.01% by weight of at least one unsaturated ω-3 C₁₆-C₂₄ fatty acid; (iii) at least about 0.01% by weight of at least one ω-6 C₁₈-C₂₂ fatty acid; and at least about 0.01% by weight of at least one ω-7 C₁₆-C₂₀ fatty acid, all of the foregoing based upon 100% total weight of the preparation.

In yet another embodiment of the present invention, the lipophilic molecule component may comprise:
about 0.1-6.0 units (by weight) palmitic acid;
about 0.1-20.0 units (by weight) linoleic acid;
about 0.1-6.0 units (by weight) alpha linolenic acid;
about 0.1-40.0 units (by weight) oleic acid;
about 0.05-8.0 units (by weight) stearic acid;
about 0.02-1.0 unit (by weight) arachidic acid;
about 0.1-0.9 units (by weight) heneicosanoic acid;
about 0.02-9.0 units (by weight) behenic acid;
about 1.0-9.0 units (by weight) tricosanoic acid;
about 0.02-9.0 units (by weight) lignoceric acid;
about 0.5-9.0 units (by weight) cerotic acid;
about 0.05-5.0 units (by weight) vaccenic acid;
about 0.02-5.0 units (by weight) gondoic acid;
about 1.0-10.0 units (by weight) heptacosanoic acid;
about 0.5-15.0 units (by weight) montanic acid;
about 2.0-26.0 units (by weight) melissic acid;
about 0.5-16.0 units (by weight) docosahexaenoic acid;
about 0.5-9.0 units (by weight) docosapentaenoic acid;
about 0.5-19.0 units (by weight) docosatetraenoic acid;
about 0.5-9.0 units (by weight) docosadienoic acid;
about 0.1-18.0 units (by weight) erucic acid;
about 0.1-0.9 units (by weight) nervonic acid;
about 10.0-800.0 units (by weight) cetyl alcohol-hexadecanol-palmityl alcohol;
about 10.0-500.0 units (by weight) 1-heptadecanol;
about 10.0-100.0 units (by weight) 1-eicosanol-arachidyl alcohol;
about 10.0-300.0 units (by weight) 1-docosanol-behenyl alcohol;
about 100.0-1500.0 units (by weight) lignoceryl alcohol-1-tetracosanol;
about 100.0-1200.0 units (by weight) 1-hexacosanol-ceryl alcohol;
about 100.0-2000.0 units (by weight) 1-heptacosanol;
about 50.0-7000.0 units (by weight) 1-octacosanol;
about 150.0-4000.0 units (by weight) 1-triacontanol-melissyl alcohol;
about 100.0-2000.0 units (by weight) dotriacontanol; and
about 500.0-1500.0 units (by weight) tetratriacontanol,
all based upon 100% total weight of the lipophilic molecule component in the preparation.

By way of additional non-limiting example, the homogenous mixture may comprise at least about 90% by weight of the vitamin C component and at least about 0.1% by weight of the lipophilic molecule component, based upon 100% total weight of the preparation. More preferably, in such an example, the homogenous mixture may comprise about 90% to about 99% by weight of the vitamin C component and from about 1% to about 8% by weight of the lipophilic molecule component. According to yet another embodiment, the homogenous mixture may comprise about 90% to about 98% by weight of the vitamin C component and from about 2% to about 7% (e.g., about 5%) by weight of the lipophilic molecule component.

According to one embodiment, the weight percentage of lipophilic molecules in the lipophilic molecule component preferably ranges from about 0.1% or 0.2% to 5.0% by weight, based upon the total weight of the preparation. By way of non-limiting example, the homogenous mixture may comprise about 0.8% to about 1.8% by weight of lipophilic molecules, and even more desirably, about 1.0% to about 1.5% by weight of lipophilic molecules, based upon the total weight of the preparation. In yet another embodiment of the present invention, the lipophilic molecule component is comprised of at least one of the following lipophilic molecules at the following weight percentages:
about 0.01-0.6% (by weight) palmitic acid;
about 0.01-2.0% linoleic acid;
about 0.01-0.6% alpha linolenic acid;
about 0.01-4.0% oleic acid;
about 0.05-2.0% stearic acid;
about 0.01-0.9% arachidic acid;
about 0.01-0.09% heneicosanoic acid;
about 0.02-0.9% behenic acid;
about 0.1-0.9% tricosanoic acid;
about 0.01-0.9% lignoceric acid;
about 0.05-0.9% cerotic acid;
about 0.05-0.5% vaccenic acid;
about 0.02-0.5% gondoic acid;
about 0.1-1.0% heptacosanoic acid;
about 0.05-1.5% montanic acid;
about 0.2-2.6% melissic acid;
about 0.05-1.6% docosahexaenoic acid;
about 0.05-0.9% docosapentaenoic acid;
about 0.05-1.9% docosatetraenoic acid;
about 0.05-0.9% docosadienoic acid;
about 0.01-1.8% erucic acid;
about 0.01-0.09% nervonic acid;
about 0.01-8.0% cetyl alcohol-hexadecanol-palmityl alcohol;
about 0.01-5.0% 1-heptadecanol;
about 0.01-1.0% 1-eicosanol-arachidyl alcohol;
about 0.01-3.0% 1-docosanol-behenyl alcohol;
about 1.0-15.0% lignoceryl alcohol-1-tetracosanol;
about 1.0-12.0% 1-hexacosanol-ceryl alcohol;
about 0.01-20.0% 1-heptacosanol;
about 0.5-70.0% 1-octacosanol;
about 15.0-40.0% 1-triacontanol-melissyl alcohol;
about 10.0-20.0% dotriacontanol; and
about 5.0-15.0% tetratriacontanol,
all based upon 100% total weight of the lipophilic molecule component in the preparation.

With regard to the lipophilic molecule component of the present invention, the lipophilic molecule component comprises lipophilic molecules which, in one embodiment of the present invention, are extracted from natural waxes and natural oils. Suitable sources of lipophilic molecules may include, but are not limited to, sugar cane wax, rice bran wax, carnauba wax, candelilla wax, Japan wax, ouricury wax, bayberry wax, shellac wax, sunflower wax, orange wax, beeswax, rice brand oil, sunflower oil, canola oil, flax seed oil, wheat germ oil, MCT oil, and hemp oil. According to one embodiment of the present invention, the set of sources of lipophilic molecules comprises rice bran wax, carnauba wax, candelilla wax, beeswax, rice brand oil, sunflower oil, canola oil, and hemp oil. According to another embodiment of the present invention, the set of sources of lipophilic molecules comprises rice bran wax, rice brand oil, sunflower oil, canola oil, and hemp oil.

With regard to the bioflavonoid component of the present invention, the bioflavonoid component comprises bioflavonoids that may include, but are not limited to: rutin, naringin, hesperidin, neohesperidin, neohesperidin dihydrochalcone, naringenin, hersperitin, nomilin, and gallic acid. According to one embodiment of the present invention, the bioflavonoid component comprises hesperidin, gallic acid, and optionally other bioflavonoids. According to another embodiment of the present invention, the bioflavonoid component comprises hesperidin, gallic acid, and optionally other bioflavonoids. By way of non-limiting example, the bioflavonoid component may comprise:
about 20-120 ppm rutin;
about 25-100 ppm naringin;
about 7000-50000 ppm hesperidin;
about 5-100 ppm neohesperidin;
about 10-100 ppm neohesperidin dihydrochalcone;
about 5-100 ppm naringenin;
about 50-600 ppm hesperitin;
about 5-100 ppm eriocitrin;
about 25-13000 ppm didymin;
about 50-150 ppm nomilin; and
about 120-1000 mg gallic acid per gram of bioflavonoids.

By way of additional non-limiting example, the bioflavonoid component may comprise:
about 20-120 ppm rutin;
about 5-100 ppm naringin;
about 7000-50000 ppm hesperidin;
about 5-100 ppm neohesperidin;
about 10-100 ppm neohesperidin dihydrochalcone;
about 5-100 ppm naringenin;
about 50-600 ppm hesperitin;
about 5-100 ppm eriocitrin;
about 25-13000 ppm didymin;
about 50-150 ppm nomilin; and
about 120-1000 mg gallic acid per gram of bioflavonoids.

In some embodiments of the present invention, the preparations and/or formulations-which may comprise controlled release formulations, sustained release implants, healthy beverages, foodstuffs, dietary supplements, topical compositions, and a wide variety of food applications-themselves may comprise a combination of active ingredients that can be administered to both humans and animals.

In other embodiments of the present invention, the vitamin C preparation can be administered to a human subject(s) to improve the status of a health condition, such as (i) promoting a healthy nervous system; (ii) preventing or decreasing the risk of developing a neurodegenerative disease; (iii) enhancing NGF-mediated neurite outgrowth; (iv) promoting wound healing; (v) enhancing fibroblast adhesion to and the interaction with the extracellular matrix; (vi) protecting the immune system from xenobiotics; (vii) decreasing the risk of developing an oxidative pathogenesis; and (viii) decreasing the risk of developing cancer, cardiovascular diseases, respiratory infections, pulmonary diseases, lung infections, atherosclerosis, respiratory diseases, and other age-related diseases associated with cytotoxic, genotoxic, and proinflammatory mechanisms. In such embodiments, desired results may be achieved by first identifying one of the above-referenced health conditions (e.g., nervous system health) and implicitly recognizing the efficacy of the present invention to accomplish one of the above-referenced purposes (e.g., promoting a healthy nervous system) and second, after such identification and implicit recognition, attending to the administration of an effective dose of the present invention to the appropriate human subject.

These and other objects, features and advantages of the present invention will become clearer when the drawings as well as the detailed description are taken into consideration.

### BRIEF DESCRIPTION OF THE DRAWINGS

For a fuller understanding of the nature of the present invention, reference should be had to the following detailed description taken in connection with the accompanying drawings in which:

FIG. 1 is a schematic, side perspective view of one embodiment of the vitamin C preparation invention.

FIG. 2 is a schematic, top perspective view of one embodiment of the homogenous mixture.

FIG. 3 is a schematic, top perspective view of another embodiment of the homogenous mixture.

FIG. 4 is a graph of the concentration of vitamin C in H9 human T-cells as measured 15-120 minutes by the procedure described in Example 2 following administration of the vitamin C preparation of Example 1 (PWC), ascorbic acid (AA), calcium ascorbate (CaA), or calcium ascorbatecalcium threonate-dehydroascorbate (Ester-C).

FIG. 5 is a graph of the percentage inhibition of 1,1-diphenyl-2-picryl hydrazyl (DPPH) reduction as measured by the procedure described in Example 4 following administration of 1, 2.5, 5, 10, or 20 g/ml of the vitamin C preparation of Example 1.

FIG. 6 is a graph of the percentage of cells exhibiting neurite outgrowth over 24 hours following administration of vehicle (or 0.5 M of the vitamin C preparation of Example 1 (PWC), ascorbic acid (AA), calcium ascorbate (CaA), or calcium ascorbatecalcium threonate-dehydroascorbate (Ester-C) or a control, as measured by the procedure described in Example 5.

FIG. 7 is a graph showing the percentage of fibroblasts adhered to fibronectin substrates following administration of vehicle (or 50 M of the vitamin C preparation of Example 1 (PWC), ascorbic acid (AA), calcium ascorbate (CaA), or calcium ascorbate-calcium threonate-dehydroascorbate (Ester-C) or as a control as measured by the procedure described in Example 6.

FIG. 8 is a graph of the plasma C-reactive protein levels in humans before and after supplementation over 24 hours following administration of the vitamin C preparation of Example 1 (PWC), ascorbic acid (AA), calcium ascorbate (CaA), or calcium ascorbatecalcium threonate-dehydroascorbate (Ester-C), as measured by the procedure described in Example 7.

FIG. 9 is a graph of the plasma oxidized low density lipoprotein (oxLDL) levels in humans before and after supplementation over 24 hours following administration of the vitamin C preparation of Example 1 (PWC), ascorbic acid (AA), calcium ascorbate (CaA), or calcium ascorbatecalcium threonate-dehydroascorbate (Ester-C), as measured by the procedure described in Example 7.

FIG. 10 is a graph of the concentration of serum vitamin C in human at various times prior (0 hours) and post supplementation as measured 1, 2, 4, 6 and 24 hours by the procedure described in Example 8, following administration of the vitamin C preparation of Example 1 (PWC), ascorbic acid (AA), calcium ascorbate (CaA), or calcium ascorbatecalcium threonate-dehydroascorbate (Ester-C).

Like reference numerals refer to like parts throughout the several views of the drawings.

### DETAILED DESCRIPTION OF THE INVENTION

Before describing the present invention in detail, it is to be understood that unless otherwise indicated this invention is not limited to specific manufacturing methods, formulation components, dosage regimens pharmaceutical preparations, delivery systems or the like, as such may vary. In advance of the foregoing, the term “about” can be defined as what one skilled in the art would understand “about” to mean, and the term “about” includes a 5% tolerance on both lower and upper bounds, if applicable.

The invention now will be described more fully hereinafter with reference to the accompanying drawings in which illustrative embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art.

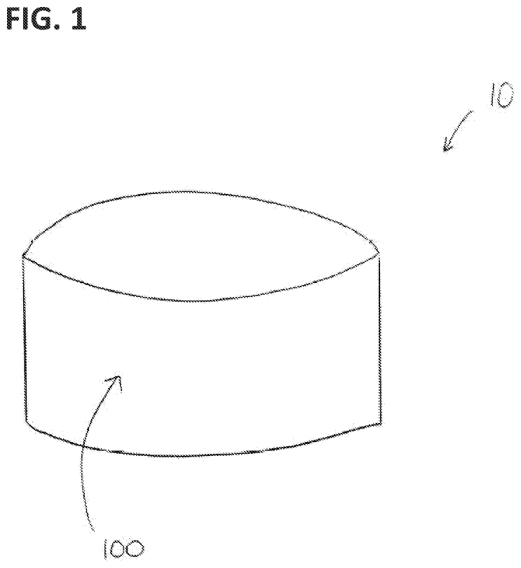

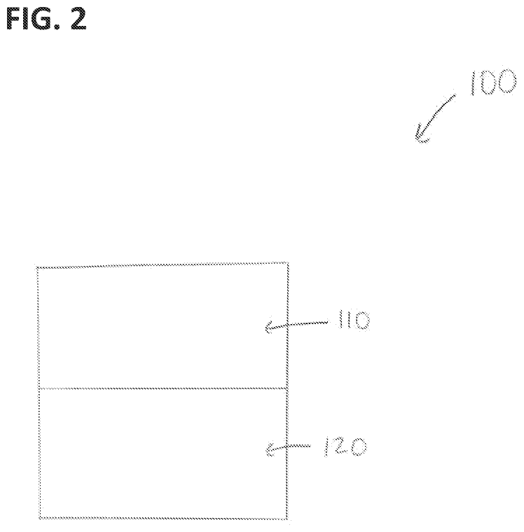

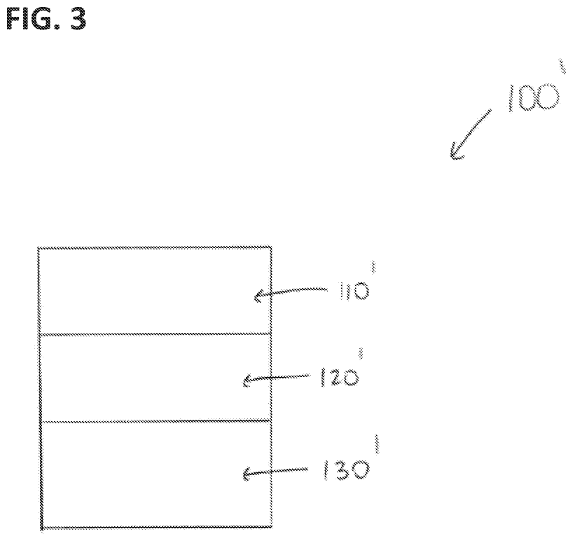

Turning now descriptively to the figures, FIGS. 1 and 2 illustrate one embodiment of an inventive vitamin C preparation which enhances absorption of vitamin C into cells and prolongs the retention of vitamin C within the blood plasma and tissue of mammals, such as humans. FIG. 3, on the other hand, illustrates another embodiment of the inventive vitamin C preparation.

FIGS. 1 and 2 show that the vitamin C preparation 10 is primarily comprised of a homogenous mixture 100, which itself is comprised of a vitamin C component 110 and a lipophilic molecule component 120. In one embodiment of the present invention, illustrated in FIG. 3, the homogenous mixture 100′ is primarily comprised of a vitamin C component 110′, a lipophilic molecule component 120′, and a bioflavonoid component 130′.

In more specific terms, the vitamin C preparation 10—preferably formulated as an oral dosage form, such as a tablet, capsule, softgel, or gummy-is comprised of a homogenous mixture 100 which is further comprised of a vitamin C component 110 and a lipophilic molecule component 120. The vitamin C component 110 of the homogenous mixture 100 comprises at least about 90% by weight of the total weight of the preparation 10. In one embodiment of the present invention, the vitamin C component comprises about 0.01 g to 2.0 g of vitamin C. The lipophilic molecule component 120, on the other hand, comprises at least about 0.1% by weight of the total weight of the preparation 10. The lipophilic molecule component 120 further comprises at least one saturated straight C₁₆-C₃₄ fatty alcohol and at least one unsaturated ω-9 C₁₈-C₂₄ fatty acid. By way of non-limiting example, the lipophilic molecule component may further comprise a combination of at least one of the following: (i) at least one saturated straight C₁₄-C₂₄ fatty acid; (ii) at least one unsaturated ω-3 C₁₆-C₂₄ fatty acid; (iii) at least one unsaturated ω-6 C₁₈-C₂₂ fatty acid; and (iv) at least one unsaturated ω-7 C₁₆-C₂₀ fatty acid. According to one embodiment of the present invention, the homogenous mixture comprises a weight ratio of vitamin C to lipophilic molecules ranging from about 1000:1 to about 10:1. According to another embodiment, the weight ratio ranges from about 100:1 to about 8:1.

In one embodiment of the present invention, the homogenous mixture may comprise a vitamin C component, a lipophilic molecule component, and a bioflavonoid component. In such an embodiment, the bioflavonoid component comprises at least about 0.1% by weight of the total weight of the preparation. By way of non-limiting example, the homogenous mixture may comprise at least about 90% by weight of the vitamin C component, at least about 0.1% to 5% by weight of the lipophilic molecule component, and at least about 0.1% to 5% by weight of the bioflavonoid component. By way of another non-limiting example, the homogenous mixture may comprise at least about 90% by weight of the vitamin C component, at least about 0.1% to 9% by weight of the lipophilic molecule component, and at least about 0.1% to 5% by weight of the bioflavonoid component.

In another embodiment of the present invention, the homogenous mixture may comprise at least about 0.1% by weight of at least one saturated straight C₁₆-C₃₄ fatty alcohol and at least about 0.01% by weight of one of at least one unsaturated ω-9 C₁₈-C₂₄ fatty acid, based upon 100% total weight of the preparation. By way of non-limiting example, the homogenous mixture may further comprise a combination of at least one of the following: (i) at least about 0.01% by weight of at least one saturated straight C₁₄-C₂₄ fatty acid; (ii) at least about 0.01% by weight of at least one unsaturated ω-3 C₁₆-C₂₄ fatty acid; (iii) at least about 0.01% by weight of at least one ω-6 C₁₈-C₂₂ fatty acid; and at least about 0.01% by weight of at least one ω-7 C₁₆-C₂₀ fatty acid, all of the foregoing based upon 100% total weight of the preparation.

In yet another embodiment of the present invention, the lipophilic molecule component may comprise:
about 0.1-6.0 units (by weight) palmitic acid;
about 0.1-20.0 units (by weight) linoleic acid;
about 0.1-6.0 units (by weight) alpha linolenic acid;
about 0.1-40.0 units (by weight) oleic acid;
about 0.05-8.0 units (by weight) stearic acid;
about 0.02-1.0 unit (by weight) arachidic acid;
about 0.1-0.9 units (by weight) heneicosanoic acid;
about 0.02-9.0 units (by weight) behenic acid;
about 1.0-9.0 units (by weight) tricosanoic acid;
about 0.02-9.0 units (by weight) lignoceric acid;
about 0.5-9.0 units (by weight) cerotic acid;
about 0.05-5.0 units (by weight) vaccenic acid;
about 0.02-5.0 units (by weight) gondoic acid;
about 1.0-10.0 units (by weight) heptacosanoic acid;
about 0.5-15.0 units (by weight) montanic acid;
about 2.0-26.0 units (by weight) melissic acid;
about 0.5-16.0 units (by weight) docosahexaenoic acid;
about 0.5-9.0 units (by weight) docosapentaenoic acid;
about 0.5-19.0 units (by weight) docosatetraenoic acid;
about 0.5-9.0 units (by weight) docosadienoic acid;
about 0.1-18.0 units (by weight) erucic acid;
about 0.1-0.9 units (by weight) nervonic acid;
about 10.0-800.0 units (by weight) cetyl alcohol-hexadecanol-palmityl alcohol;
about 10.0-500.0 units (by weight) 1-heptadecanol;
about 10.0-100.0 units (by weight) 1-eicosanol-arachidyl alcohol;
about 10.0-300.0 units (by weight) 1-docosanol-behenyl alcohol;
about 100.0-1500.0 units (by weight) lignoceryl alcohol-1-tetracosanol;
about 100.0-1200.0 units (by weight) 1-hexacosanol-ceryl alcohol;
about 100.0-2000.0 units (by weight) 1-heptacosanol;
about 50.0-7000.0 units (by weight) 1-octacosanol;
about 150.0-4000.0 units (by weight) 1-triacontanol-melissyl alcohol;
about 100.0-2000.0 units (by weight) dotriacontanol; and
about 500.0-1500.0 units (by weight) tetratriacontanol, all based upon 100% total weight of the lipophilic molecule component in the preparation.

By way of additional non-limiting example, the homogenous mixture may comprise at least about 90% by weight of the vitamin C component and at least about 0.1% by weight of the lipophilic molecule component, based upon 100% total weight of the preparation. More preferably, in such an example, the homogenous mixture may comprise about 90% to about 99% by weight of the vitamin C component and from about 1% to about 8% by weight of the lipophilic molecule component. According to yet another embodiment, the homogenous mixture may comprise about 90% to about 98% by weight of the vitamin C component and from about 2% to about 7% (e.g., about 5%) by weight of the lipophilic molecule component.

According to one embodiment, the weight percentage of lipophilic molecules in the lipophilic molecule component preferably ranges from about 0.1% or 0.2% to 5.0% by weight, based upon the total weight of the preparation. By way of non-limiting example, the homogenous mixture may comprise about 0.8% to about 1.8% by weight of lipophilic molecules, and even more desirably, about 1.0% to about 1.5% by weight of lipophilic molecules, based upon the total weight of the preparation. In yet another embodiment of the present invention, the lipophilic molecule component is comprised of at least one of the following lipophilic molecules at the following weight percentages:
about 0.01-0.6% (by weight) palmitic acid;
about 0.01-2.0% linoleic acid;
about 0.01-0.6% alpha linolenic acid;
about 0.01-4.0% oleic acid;
about 0.05-2.0% stearic acid;
about 0.01-0.9% arachidic acid;
about 0.01-0.09% heneicosanoic acid;
about 0.02-0.9% behenic acid;
about 0.1-0.9% tricosanoic acid;
about 0.01-0.9% lignoceric acid;
about 0.05-0.9% cerotic acid;
about 0.05-0.5% vaccenic acid;
about 0.02-0.5% gondoic acid;
about 0.1-1.0% heptacosanoic acid;
about 0.05-1.5% montanic acid;
about 0.2-2.6% melissic acid;
about 0.05-1.6% docosahexaenoic acid;
about 0.05-0.9% docosapentaenoic acid;
about 0.05-1.9% docosatetraenoic acid;
about 0.05-0.9% docosadienoic acid;
about 0.01-1.8% erucic acid;
about 0.01-0.09% nervonic acid;
about 0.01-8.0% cetyl alcohol-hexadecanol-palmityl alcohol;
about 0.01-5.0% 1-heptadecanol;
about 0.01-1.0% 1-eicosanol-arachidyl alcohol;
about 0.01-3.0% 1-docosanol-behenyl alcohol;
about 1.0-15.0% lignoceryl alcohol-1-tetracosanol;
about 1.0-12.0% 1-hexacosanol-ceryl alcohol;
about 0.01-20.0% 1-heptacosanol;
about 0.5-70.0% 1-octacosanol;
about 15.0-40.0% 1-triacontanol-melissyl alcohol;
about 10.0-20.0% dotriacontanol; and
about 5.0-15.0% tetratriacontanol,
all based upon 100% total weight of the lipophilic molecule component in the preparation.

With regard to the lipophilic molecule component 120 of the present invention, the lipophilic molecule component 120 comprises lipophilic molecules which, in one embodiment of the present invention, are extracted from natural waxes and natural oils for increased lipophilicity. The lipophilic molecules comprised within the lipophilic molecule component 120 are termed “absorption promoters” and are vital components of cellular membranes (and are involved in the communication among individual cells), are pivotal in the regulation and control of cellular function, and functionally enhance entry into cells and transport via the body's active transport and simple diffusion-dependent pathways. These molecules act as vitamin C carriers to increase the intestinal absorption and tissue distribution of vitamin C and enhance cellular uptake kinetics, which allows for vitamin C to enter cells quicker and in a more safe and effective manner. Lipophilic molecules can shuttle between a number of different pathways, and the effects of increased lipophilicity include a mechanism for enhancing the uptake, distribution, and release kinetics; favorably affecting the volume of distribution; and ease of interaction with cells in a non-toxic way. Furthermore, lipophilic molecules enhance the elimination of electrophilic lipid peroxidation products (in the ascorbylation pathway) and have implications for the prevention of chronic inflammatory diseases and oxidative stress. Suitable sources of lipophilic molecules may include, but are not limited to, sugar cane wax, rice bran wax, carnauba wax, candelilla wax, Japan wax, ouricury wax, bayberry wax, shellac wax, sunflower wax, orange wax, beeswax, rice brand oil, sunflower oil, canola oil, flax seed oil, wheat germ oil, MCT oil, and hemp oil. According to one embodiment of the present invention, the set of sources of lipophilic molecules comprises rice bran wax, carnauba wax, candelilla wax, beeswax, rice brand oil, sunflower oil, canola oil, and hemp oil. According to another embodiment of the present invention, the set of sources of lipophilic molecules comprises rice bran wax, rice brand oil, sunflower oil, canola oil, and hemp oil.

The lipophilic molecules which comprise the lipophilic molecule component 120 can be obtained by (1) saponification of a wax or oil (e.g., a natural wax and natural oil), (2) solidifying and grinding the saponified wax or oil to a d₉₀ less than 2000 microns (e.g., 100-500 microns or 500-2000 microns), (3) extracting the ground material with acetone or an alcohol (e.g., ethanol or isopropanol), and (4) optionally solidifying and grinding the extracted material to a d₉₀ less than microns (e.g., 100-500 microns or 500-2000 microns). To this point, the method used to extract lipophilic molecules from the natural waxes and natural oils tremendously affects the ability of the vitamin C to be absorbed into the body of the cellular compartments to increase their bioactivity and collective health. The process of continuous-lipid-extraction of lipophilic molecules produces a superiorly absorbed form of vitamin C has enhanced delivery, availability, absorption kinetics, distribution, uptake, concentration, and utilization efficacy of essential vitamin C in the human body. The natural waxes and natural oils undergo saponification or hydrolysis before the extraction procedure. For saponification, the natural waxes and natural oils are heated using a jacketed kettle at 90° C. for 3 hours until the waxes and oils completely melted, KOH is added, and the mixture is held at 90° C. for 1 hour with stirring. For hydrolysis, the natural waxes and natural oils are heated using a jacketed kettle at 90° C. for 3 hours until the wax and oil completely melted, aqueous sulfuric acid solution is added, and mixture is held at 90° C. for 1 hour with stirring. After 1 hour of stirring, the saponified orhydrolyzed wax and oil is poured into cart trays and dried at 21.1° C. before undergoing the extraction procedure.

The extraction of natural waxes and natural oils may be performed by either dispersed-solids extraction or immersion type percolation extraction. For the dispersed-solids extraction, the natural waxes and natural oils are ground to a particle mesh size of 100 to 425 microns and subjected to liquid extraction in a dispersed-solids extraction system. In the case of immersion type percolation extraction, the natural waxes and natural oils are ground to a particle mesh size of 500 to 2000 microns and subjected to liquid extraction in a solid-liquid immersion type percolating extractor system. In both types of extraction equipment, the natural mixture of aliphatic alcohols, saturated fatty acids, and omega-3, omega-6, omega-7, omega-9 fatty acids is selectively extracted with adequate hot organic solvents such as acetone and ethanol with a temperature range of about 55° C. to 75° C. The extractions are purified with hot organic solvents such as hexane, heptane, and acetone; recovered; and dried. The extractions contain a mixture of aliphatic alcohols having 16 to 34 carbon atoms, saturated fatty acids having 16 to 30 saturated carbon atoms, and omega-3, omega-6, omega-9 fatty acids with melting point between about 70° C. to 80° C. The ratio of the natural wax and natural oil particles to hot liquid extractants is from about 1 to 4 and about 1 to 10. According to one embodiment of the present invention, the extractions with hot organic solvents occur at about 60° C., and the ratio of natural wax and natural oil particles to hot liquid extractants is about 1 to 8.

With regard to the bioflavonoid component 130′ of the present invention, the bioflavonoid component 130′ comprises bioflavonoids that may include, but are not limited to: rutin, naringin, hesperidin, neohesperidin, neohesperidin dihydrochalcone, naringenin, hersperitin, nomilin, and gallic acid. According to one embodiment of the present invention, the bioflavonoid component comprises hesperidin, gallic acid, and optionally other bioflavonoids. According to another embodiment of the present invention, the bioflavonoid component comprises hesperidin, gallic acid, and optionally other bioflavonoids. By way of non-limiting example, the bioflavonoid component may, for purposes of protecting the vitamin C preparation from oxidizers and contributing to the preparation's antioxidant capabilities, comprise:
about 20-120 ppm rutin;
about 25-100 ppm naringin;
about 7000-50000 ppm hesperidin;
about 5-100 ppm neohesperidin;
about 10-100 ppm neohesperidin dihydrochalcone;
about 5-100 ppm naringenin;
about 50-600 ppm hesperitin;
about 5-100 ppm eriocitrin;
about 25-13000 ppm didymin;
about 50-150 ppm nomilin; and
about 120-1000 mg gallic acid per gram of bioflavonoids.

By way of additional non-limiting example, the bioflavonoid component may, for purposes of protecting the vitamin C preparation from oxidizers and contributing to the preparation's antioxidant capabilities, comprise:
about 20-120 ppm rutin;
about 5-100 ppm naringin;
about 7000-50000 ppm hesperidin;
about 5-100 ppm neohesperidin;
about 10-100 ppm neohesperidin dihydrochalcone;
about 5-100 ppm naringenin;
about 50-600 ppm hesperitin;
about 5-100 ppm eriocitrin;
about 25-13000 ppm didymin;
about 50-150 ppm nomilin; and
about 120-1000 mg gallic acid per gram of bioflavonoids.

In some embodiments of the present invention, the preparations and/or formulations-which may comprise controlled release formulations, sustained release implants, healthy beverages, foodstuffs, dietary supplements, topical compositions, and a wide variety of food applications-themselves may comprise a combination of active ingredients that can be administered to both humans and animals.

In other embodiments of the present invention, the vitamin C preparation can be administered to a human subject(s) to improve the status of a health condition, such as (i) promoting a healthy nervous system; (ii) preventing or decreasing the risk of developing a neurodegenerative disease; (iii) enhancing NGF-mediated neurite outgrowth; (iv) promoting wound healing; (v) enhancing fibroblast adhesion to and the interaction with the extracellular matrix; (vi) protecting the immune system from xenobiotics; (vii) decreasing the risk of developing an oxidative pathogenesis; and (viii) decreasing the risk of developing cancer, cardiovascular diseases, respiratory infections, pulmonary diseases, lung infections, atherosclerosis, respiratory diseases, and other age-related diseases associated with cytotoxic, genotoxic, and proinflammatory mechanisms. In such embodiments, desired results may be achieved by first identifying one of the above-referenced health conditions (e.g., nervous system health) and implicitly recognizing the efficacy of the present invention to accomplish one of the above-referenced purposes (e.g., promoting a healthy nervous system) and second, after such identification and implicit recognition, attending to the administration of an effective dose of the present invention to the appropriate human subject.

### Dosage Forms

The vitamin C preparation is preferably in the form of an oral dosage form, such as beads, pellets, granules, capsules (soft or hard), sachets, tablets, powders, dispersible powders capable of effervescing upon addition of water, aqueous or oily suspensions, emulsions, syrups, elixirs, or lozenges. For example, the oral dosage form can be a chewable tablet or gum; oral liquid dosage form, such as a suspension in an aqueous or non-aqueous liquid solution; or an emulsion, which can be a soft drink, tea, milk, coffee, juice, sports drink, or water. In some embodiments, the vitamin C preparation is incorporated into various products, such as nutritional supplements (including vitamins and multivitamins), foods (including health food products such as nutrition bars), and drinks (including fruit juices such as energy drinks).

The compositions, preparations, formulations, combinations, conjugations of the invention are suitable for oral administration and may be presented as solid dosage form units such as beads, pellets, granules, capsules, sachets, tablets, powders, powders capable of effervescing upon addition of water or suspensions and/or lozenges, each containing a predetermined amount of the active compound, although any pharmaceutically acceptable dosage form can be utilized, and also the compositions for oral administration may be presented as a liquid dosage forms such as pharmaceutically acceptable suspensions in aqueous liquors or non-aqueous liquids such as syrup an elixir, solution, or an emulsion, and also in soft drinks, tea, milk, coffee, juices, sports drinks and water. The compositions, preparations, formulations, combinations, conjugations of the invention are suitable also for topical use in conventional forms such as solutions, suspensions, lotions, emulsions, ointments, creams and gels.

Generally, the daily dosage of most embodiments of the vitamin C preparation on a vitamin C weight basis can range from about 30 mg to 2 g. For instance, in one such embodiment, the daily dosage can be about 60 mg to 1 g or about 60 mg to about 500 mg. In a preferred embodiment, the daily dosage ranges from about 60 mg to about 500 mg (e.g., the daily dosage can be 400 mg). According to another embodiment, the daily dosage ranges from about 60 mg to about 200 mg (e.g., the daily dosage can be 60, 100, or 200 mg). In some embodiments, the daily dose can be achieved by administration of a single dosage form of the present invention or, alternatively, two or more such dosage forms. In a preferred embodiment, the daily dose is achieved by administration of only one or two dosage forms (e.g., once daily dosing or b.i.d.). Therefore, the present invention may include, but is not limited to, dosage forms containing 30, 60, 100, 200, 400, 500, or 1000 mg of the vitamin C preparation (on a vitamin C weight basis).

Some embodiments of the vitamin C preparation may also include one or more excipients or additives. Suitable excipients and additives may include, but are not limited to, additional antioxidants (e.g., phenolic compounds), inert diluents (such as lactose, sodium carbonate, calcium phosphate, and calcium carbonates), granulating and disintegrating agents (such as corn starch and algenic acid), binders (such as starch), lubricants (such as magnesium stearate, stearic acid and talc), preservatives (such as ethyl or propyl p-hydroxybenzoate), colorants, flavoring agents, release modifying agents, thickeners, and any combination of any of the foregoing. Suitable antioxidants may include, but are not limited to, bioflavonoids, flavonoids, flavonols, flavanones, flavones, flavonals, flavanolols, and flavanols.

Some embodiments of the present invention can also contain other components useful in formulating pharmaceutical preparations for administration to humans and animals, including swelling agents, surfactants, solvents, preservatives, inert diluents, stabilizers, granulating agents, buffers, lubricants, disintegrating agents, antioxidants, coating agents, binders and the like, all of which are standard in the pharmaceutical arts. Suitable inert solid diluents may comprise calcium carbonate, calcium phosphate and kaolin. Suitable diluents for soft capsules include, but are not limited to, water and oils such as peanut oil, liquid paraffin, corn oil, wheat germ oil, soybean oil, and olive oil.

In some embodiments of the present invention, aqueous suspensions or dispersions contain the vitamin C preparation, for example, in fine powder form together with one or more suspension or dispersion (or wetting) agents. Suitable suspension agents may include, but are not limited to, sodium carboxymethylcellulose, methylcellulose, hydroxypropylmethylcellulose, sodium alginate, polyvinyl-pyrrolidone, gum tragacanth and gum acacia. Suitable dispersing or wetting agents may include, but are not limited to, lecithin, condensation products of an alkylene oxide with fatty acids, condensation products of ethylene oxide with long chain aliphatic alcohols, condensation products of ethylene oxide with partial esters derived from fatty acids and a hexitol such as polyoxyethylene sorbitol monooleate, or condensation products of ethylene oxide with partial esters derived from fatty acids and hexitol anhydrides.

Dispersible powders and granules suitable for preparation of an aqueous suspension by the addition of water contain the vitamin C preparation, for example, together with a dispersing agent, wetting agent, or suspending agent. Suitable dispersing agents, wetting agents, and suspending agents include those mentioned above.

In some embodiments, oily suspensions may be formulated by suspending the vitamin C preparation in an oil, such as a vegetable oil or a mineral oil. The oily suspensions may also contain a thickening agent such as carnauba wax, candelilla wax, rice bran wax, beeswax, hard paraffin, or cetyl alcohol.

The vitamin C preparation may, in some embodiments, be in the form of an oil-in-water emulsion. The oily phase may be a vegetable-based oil or a mineral-based oil. Suitable emulsifying agents may include, but are not limited to, naturally occurring gums (such as acacia and tragacanth gum), naturally occurring phosphatides (such as soybean, lecithin, esters, and partial esters derived from fatty acids and hexitol anhydrides) and condensation products of partial esters with ethylene oxide (such as polyoxyethylene sorbitan monooleate).

Syrups and elixirs may be formulated with sweetening agents such as glycerol, propylene glycol, sorbitol, aspartame, or sucrose, and may also contain a demulcent, preservative, flavoring, or coloring agent.

In other embodiments, the vitamin C preparation may be in a form suitable for administration by inhalation (e.g., as a finely divided powder or a liquid aerosol), or for parenteral administration (e.g., as a sterile aqueous or oily solution for intravenous, subcutaneous, intramuscular dosing or as a suppository for rectal dosing). Administration of the vitamin C preparation by these non-oral routes is advantageous, as administration by these methods avoids gastrointestinal side effects, which may accompany high doses of vitamin C released in the stomach.

In some embodiments, the vitamin C preparation can be delivered topically, for example, to protect the skin from free radicals, promote wound healing (for instance, for healing cuts, abrasions, sun damage (e.g., sun burn), wrinkles, and scars), and/or reduce inflammation. The vitamin C preparation of the invention is advantageous compared to what is currently available, because the invention provides superior penetration of vitamin C through the skin than vitamin C alone can accomplish. Transdermal delivery of the vitamin C preparation permits systemic delivery of the vitamin C while avoiding gastrointestinal side effects. In some embodiments, the topical formulation containing the vitamin C preparation can be in the form of a solution, suspension, lotion, emulsion, ointment, cream, or gel. According to one embodiment of the present invention, the topical formulation is a cream or lotion. The formulation may include additional active ingredients. These formulations may be prepared by methods known in the art, and typically include a topically acceptable vehicle. One embodiment is a topical formulation containing about 0.5 to about 25% by weight of the vitamin C preparation of the present invention, based upon 100% total weight of the topical formulation. For instance, the topical formulation can contain 0.5-2%, 1-2%, 1-5%, 1-10%, 5-15%, 5-20%, or 10-20% by weight of the vitamin C preparation.

In one embodiment of the present invention, the vitamin C preparation is used to coat a medical device that is then positioned to a desired target location within the body, whereupon the vitamin C preparation elutes from the medical device. In such an embodiment, the coating preferably includes a therapeutically effective amount of the vitamin C preparation. In another embodiment, the medical device is positioned so that the vitamin C preparation is released in a therapeutically effective amount to a targeted site, such as a diseased or injured tissue or organ. The device can be introduced temporarily or permanently into a mammal (e.g., a human) for the prophylaxis or therapy of a medical condition, or to augment the immune system. The device can be introduced subcutaneously, percutaneously, or surgically. The medical device can be selected from stents, synthetic grafts, artificial heart valves, artificial hearts, and fixtures to connect the prosthetic organ to the vasculature, venous valves, abdominal aortic aneurysm grafts, inferior venal caval filters, catheters including permanent drug infusing catheters, embolic coils, embolic materials used in vascular embolization mesh repair materials, a Dracon vascular particle orthopedic metallic plates, rods, screws, and vascular sutures.

The vitamin C preparation may be formulated to provide immediate release or controlled release (e.g., sustained release) of the vitamin C preparation, for example, to provide effective doses of vitamin C over extended periods of time to prolong the biological activity and beneficial biochemical functions of vitamin C. One embodiment of the invention is a controlled release dosage form (such as a solid dosage form) containing about 0.01 g to 2.0 g of vitamin C, about 1 mg to 100 mg of lipophilic molecules, and about 1 mg to 500 mg of bioflavonoids. For example, the controlled release dosage form may release about 10 to about 35% by weight of the total vitamin C preparation within about 2 hours in an in vitro dissolution test, and about 40 to about 70% by weight of the total vitamin C preparation within about 8 hours. According to another embodiment, the controlled release dosage form may release about 50% by weight of the total vitamin C preparation within about 2 hours in an in vitro dissolution test, and more than 90% by weight of the total vitamin C preparation within about 6 or 8 hours. Any type of controlled release system known in the art can be used. The in vitro dissolution test is conducted using the Basket Method (Apparatus 1) with 900 ml 0.1M HCl as the medium run at 100 RPM at a temperature of 37° C. The samples are filtered through Whatman filter paper #1 and the amount of vitamin C is calculated based on the equivalence to standard dicholorophenol-indophenol solutions. Solid controlled release dosage forms (e.g., tablets) can be formulated (e.g., coated) so as to prolong the release of the vitamin C preparation into the gastrointestinal tract, or to prevent the release of the vitamin C preparation in the stomach in order to prevent or attenuate the gastrointestinal side effects which can accompany high doses of vitamin C released in the stomach. For example, the vitamin C preparation can be enteric coated so as to prevent significant release of the preparation in the stomach. Controlled release of the vitamin C preparation can prolong therapeutic and/or immunoprotective systemic concentrations of vitamin C in a person.

One embodiment of the invention is a three-layer controlled release dosage form (e.g., a tablet) where each layer contains a vitamin C preparation of the invention. The vitamin C preparation of each layer can be the same or different. At least one of the layers provides controlled release of the vitamin C preparation. For example, the dosage form can include (i) a first layer, (ii) a second layer, and (iii) an outer layer surrounding the first and second layers, where the first layer and outer layer provide controlled release of the vitamin C preparation(s) and the second layer provides immediate release of the vitamin C preparation. According to one variation of this embodiment, the outer layer releases substantially all (>90%) of the vitamin C preparation in a controlled manner within 60, desirably 30, and even more desirably 20 minutes, as determined by the aforementioned in vitro dissolution test. The second layer provides immediate release of the vitamin C preparation contained therein. Finally, the first layer releases the vitamin C preparation contained therein in a controlled manner over at least 6 hours (e.g., substantially of the vitamin C preparation may be released within 6-10 hours or 6-8 hours), as determined by the aforementioned in vitro dissolution test.

Transdermal patch devices can also provide controlled administration (e.g., continuous or other sustained administration) of the vitamin C preparation. Methods for preparing controlled release transdermal formulations are known in the art. For example, the transdermal device may contain an impermeable backing layer which defines the outer surface of the device and a permeable skin attaching membrane, such as an adhesive layer, sealed to the outer layer in such a way as to create a reservoir between them wherein the therapeutic agent is placed (e.g., a bandage or patch (including a time released patch)).

Other suitable controlled release systems may include, but are not limited to, long-term sustained implants, aqueous or oily suspensions, emulsions, syrups, elixirs, or lozenges, chewable tablet or gum, foods, beverages, osmotic systems, and dissolution system (e.g., effervescent oral dosage form).

The vitamin C preparation of the present invention is preferably administered orally to a mammal (e.g., a human), but it can also be administered by other routes of administration, such as intravenously or subcutaneously.

### Preparation of Formulation

The vitamin C preparation of the present invention may be prepared by methods wellknown in the art, such as mixing the vitamin C, lipophilic molecules, optionally bioflavonoids, and any desired excipients. The following examples illustrate the invention without limitation.

### Example 1

### Lipid Metabolite Extraction:

Saponification: 25 kg of rice bran wax was heated using a jacketed kettle at 90° C. for 3 hours until the wax was completely melted. 4.67 L of 8.0 M KOH (450 g/l) in water was slowly added with continuous stirring and heating. The mixture was held at 90° C. for 1 hour with stirring. After 1 hour the saponified wax was poured into cart trays and dried at 21.1° C. The 32.1 kg of cooled dried saponified wax was then ground to a powder (100-425 or 500-2000 microns).

Extraction: 9.6 kg of the saponified wax was placed in 8 extraction thimbles (1.2 kg of saponified wax per extraction thimble). 100 L of acetone were pumped into a 200 L cylindrical-bottom flask and connected to a soxhlet system. The system was refluxed for approximately 24 hours, and the extract was pumped to a jacketed reactor. The extract was chilled to approximately 10° C. with 20 rpm agitation (20 rpm) for 10 hours. The chilled extract was then centrifuged in a vertical basket centrifuge. The collected solid was poured into trays and vacuum dried for 16 hours. The dried solid was then ground to a powder.

### Preparation of the Vitamin C Preparation:

A jacketed mixer was charged with dry powder of 58 kg of vitamin C, 0.75 kg of the lipid metabolites prepared above and 1.5 kg of bioflavonoids. The mixer was then turned on (agitation is initiated—plows) to create a homogenous mixture of dry powder. The high-speed shearing devices (choppers) were initiated for 1 minute. Hot water was then pumped through the jacket of the mixer to heat the mixture to 80° C. with continuous mixing (plows only) for 15 minutes for complete encapsulation. The encapsulated mixture was cooled by running chilled water (10° C.) through the jacket under continuous mixing for 1 hour until a free-flowing powder was formed. The powder was discharged into a double polyethylene-lined container and then passed through a comminuting mill running at approximately 2500 rpm equipped with a 0.15 mm screen. The milled powder was collected into appropriately labeled, double polyethylene-lined drums and reconciled.

TABLE 1
Formulation of One Embodiment of the Present Invention

| Ingredients | Amount | |
|---|---|---|
| Vitamin C | 90-99% | |
| Lipophilic Molecules | 0.1-5% | |
| Palmitic Acid | 100-600 | mg/g* |
| Linoleic Acid (Omega-6 fatty acid) | 100-2000 | mg/g |
| Alpha Linolenic Acid (Omega-3 fatty acid) | 100-600 | mg/g |
| Oleic Acid (Omega-9 fatty acid) | 100-4000 | mg/g |
| Stearic Acid | 10-80 | mg/g |
| Arachidic Acid | 10-90 | mg/g |
| Heneicosanoic Acid | 10-90 | mg/g |
| Behenic Acid | 10-90 | mg/g |
| Tricosanoic Acid | 10-90 | mg/g |
| Lignoceric Acid | 10-90 | mg/g |
| Cerotic Acid | 10-90 | mg/g |
| Vaccenic Acid (Omega-7 fatty acid) | 10-90 | mg/g |
| Gondoic Acid | 10-90 | mg/g |
| Heptacosanoic Acid | 10-100 | mg/g |
| Montanic Acid | 50-150 | mg/g |
| Melissic Acid | 2.0-26.0 | mg/g |
| Docosahexaenoic Acid (DHA) | 50-160 | mg/g |
| (Omega -3 fatty acid) | | |
| Docosapentaenoic Acid (DPA) | 50-90 | mg/g |
| (Omega-3 fatty acid) | | |
| Docosatetraenoic Acid (DTA) | 50-190 | mg/g |
| (Omega-6 fatty acid) | | |
| Docosadienoic Acid (Omega-6 fatty acid) | 50-90 | mg/g |
| Erucic Acid (Omega -9 fatty acid) | 10-180 | mg/g |
| Nervonic Acid (Omega-9 fatty acid) | 10-90 | mg/g |
| Cetyl Alcohol - Hexadecanol - Palmityl | 10-800 | mg/g |
| Alcohol | | |
| 1- Heptadecanol | 10-500 | mg/g |
| 1- Eicosanol - Arachidyl alcohol | 10-100 | mg/g |
| 1- Docosanol - Behenyl Alcohol | 10-300 | mg/g |
| Lignoceryl Alcohol - 1- Tetracosanol | 100-1500 | mg/g |
| 1- Hexacosanol - Ceryl Alcohol | 100-1200 | mg/g |
| 1- Heptacosanol | 100-2000 | mg/g |
| 1- Octacosanol | 50-7000 | mg/g |
| 1- Triacontanol - Melissyl Alcohol | 150-4000 | mg/g |
| Dotriacontanol | 100-2000 | mg/g |
| Tetratriacontanol | 500-1500 | mg/g |
| Bioflavonoids (optional) | 0.1-5% | |
| Rutin | 20-120 | ppm** |
| Naringin | 25-100 | ppm |
| Hesperidin | 7,000-50,000 | ppm |
| Neohesperidin | 5-100 | ppm |
| Neohesperidin Dihydrochalcone | 10-100 | ppm |
| Naringenin | 5-100 | ppm |
| Hersperitin | 50-600 | ppm |
| Eriocitrin | 5-100 | ppm |
| Didymin | 25-13,000 | ppm |
| Nomilin | 50-150 | ppm |

| | |
|---|---|
| Gallic Acid | At least 120 mg/g (q.s.) |

*mg/g = mg of component per g of total lipophilic molecules
**ppm or mg/g = ppm or mg of component per g of total bioflavonoid mixture

### Example 2

The rate of vitamin C absorption in H9 cells, a human T-cell line, was determined for the formulation of Example 1 and other vitamin C formulations.

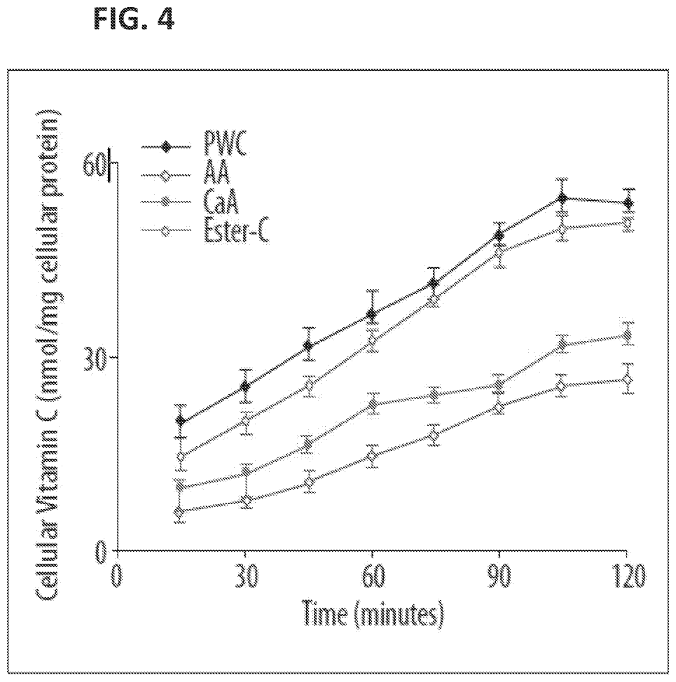

Cells from the human T-lymphoblastic H9 cell line were starved of vitamin C for 18 hours in serum-free media and subsequently suspended in 50 μM of (1) ascorbic acid (AA), (2) calcium ascorbate (CaA), (3) calcium ascorbate-calcium threonatedehydroascorbate (commercially available as Ester-C), or (4) the vitamin C preparation of Example 1 (PWC). At the times indicated in FIG. 4, cells were harvested and measured for vitamin C and protein content. The cellular vitamin C levels of the cells were measured using the 2,4-dinitrophenylhydrazine spectrophotometric technique (Bessey et al., 1947).

Over a two-hour period, the level of vitamin C uptake from Example 1 was consistently higher than that observed with ascorbic acid, calcium ascorbate, and calcium ascorbate-calcium threonate-dehydroascorbate (See FIG. 4). At fifteen minutes, cellular vitamin C levels ranged from 7±1.4 nmol/mg cellular protein with ascorbic acid, to over double that amount (15±2.4 nmol/mg protein) with the vitamin C preparation of Example 1. The absorbed vitamin C levels rose significantly with time, peaking at approximately two hours with cellular levels ranging from 31 nmol/mg protein for ascorbic acid and 50 nmol/mg protein for the vitamin C preparation of Example 1.

In order for vitamin C to exert its beneficial effects, it must be taken up into the cell. To date, vitamin-C lipid metabolites exhibit the greatest amount of vitamin C uptake and retention as compared to all other vitamin C formulations. The result leads to human T-lymphocytes having a more rapidly absorbed form of vitamin C and higher cellular levels of the same (233% higher) as compared to all forms of vitamin C tested at all time points. Results are shown in FIG. 4.

### Example 3

The ability to inhibit pesticide-induced T-lymphocyte aggregation was determined for the formulation of Example 1 and other vitamin C formulations.

The human T-lymphoblastic H9 cell line was incubated with vehicle (−) or with one of two activators of T-lymphocyte aggregation, phytohemagglutinin (PHA; 10 μm) or bifenthrin (10 mM). The cells were immediately treated with 0.5 μM of (1) ascorbic acid (AA), (2) calcium ascorbate (CaA), (3) calcium ascorbate-calcium threonatedehydroascorbate (Ester-C), or (4) the vitamin C preparation of Example 1 (PWC) for 30 minutes at 37° C. After treatment, the ability of each formulation to inhibit homotypic aggregation was measured by counting aggregate size at 400× magnification. The vitamin C preparation of Example 1 inhibited the aggregation of the T-lymphocytes induced by the pesticide PHA or the pesticide bifenthrin by 88% and 84% respectively (Table 2). The reduction in T-lymphocyte aggregation was greater following treatment with the vitamin C preparation of Example 1 than any of the other formulations.

Leukocyte cell-cell adhesion is associated with xenobiotic induced hyperactivation and inflammatory damage, and vitamin C has been shown to prevent cigarette smoke-induced leukocyte aggregation and attachment to vascular endothelium (Lehr et al., 1994; Weber et al., 1996). As shown in Table 2, vitamin C has also been shown to reduce pesticide mediated T-cell hyperactivation. Given that the formulation of the current invention has greater ability to prevent pesticide-induced T-cell aggregation than other vitamin C formulations, suggests that the formulation of the present invention will provide greater protection against other deleterious xenobiotics and increase the level of protection afforded by the immune system by a factor of at least 2.5. Furthermore, the preparation of Example 1 is more efficient than all forms of vitamin C tested at all points in time.

TABLE 2
Activators of T-Cell Aggregation
The vitamin C preparation of Example 1 inhibits xenobiotic induced
homotypic aggregation in human T-lymphocytes more effectively than
calcium ascorbate-calcium threonate-dehydroascorbate (Ester-C).
Activators of T-Cell Aggregation

| Vit. C Added | None | PHA | Bifenthrin |
|---|---|---|---|
| None | 10 ± 5 | 170 ± 15 | 300 ± 13 |
| AA | 9 ± 4 | 75 ± 12 | 130 ± 5 |
| CaA | 12 ± 4 | 110 ± 10 | 137 ± 8 |
| Ester-C | 8 ± 2 | 120 ± 17 | 200 ± 8 |
| *PWC | 11 ± 6 | 20 ± 9 | 50 ± 10 |

### Example 4

The antioxidant and free radical scavenging activity was determined for the vitamin C preparation of Example 1 and known dietary antioxidants.

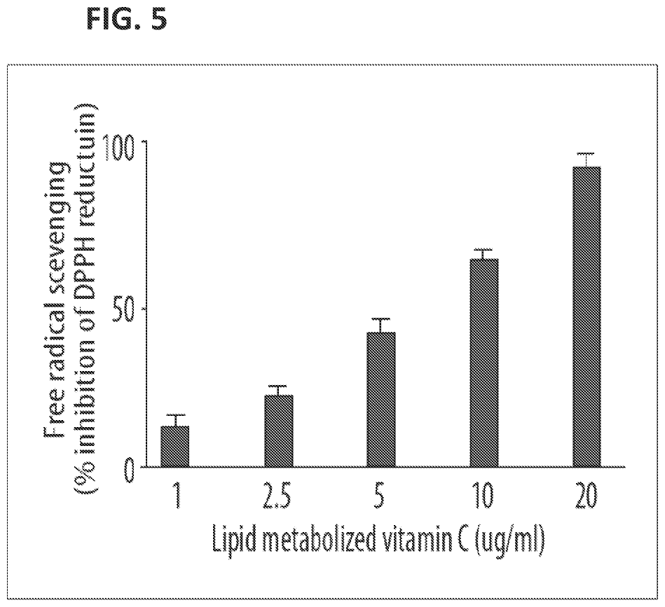

Briefly, 200 ml of a 1, 2.5, 5, 10, or 20 μg/ml solution of the vitamin C preparation of Example 1 was mixed with 50 μl of a 659 μM 1,1-diphenyl-2-picryl hydrazyl (DPPH) solution and incubated at 25° C. for 20 minutes. Free radical scavenging activity of the vitamin C preparation of Example 1 was measured by the reduction of 1,1-diphenyl-2-picryl hydrazyl (DPPH) to 1,1-diphenyl-2-picryl hydrazine at an absorbance of 510 nm. The results are shown in FIG. 5.

The vitamin C preparation dose dependently scavenged DPPH free radicals. The vitamin C preparation demonstrated excellent scavenging ability by reducing the DPPH-induced free radical concentration by 93% at its maximum concentration.

The peroxyl radical oxygen reactive species (ORAC) scavenging ability of the vitamin C preparation was also determined. The ORAC assay detects free radical damage to fluorescein induced by 2,2″-Asobix dihydrochloride (AAPH; 153 mM), and the change is measured by fluorescence spectrophotometry. Antioxidants inhibit the free radical range damage to the fluorescent compound and prevent the reduction in fluorescence. The results are shown in Table 3. The results from different concentrations of the vitamin C preparation of Example 1 were compared to the known antioxidant Trolox®. The ORAC results are expressed as Trolox® equivalents (6-Hydroxy-2,5,7,8tetramethylchroman-2-carboxylic Acid; TE) per gram of sample.

Vitamin C is a chemical reducing agent for many intracellular and extracellular reactions such as oxidative DNA or protein damage, low-density lipoprotein oxidation, lipid peroxidation, oxidants, the formation of nitrosamines in gastric juice, extracellular oxidants from neutophils, and endothelium dependent vasodilation. The vitamin C preparation of the present invention, which exhibits potent antioxidant and free radical scavenging effects in vitro, can serve as a good vitamin C preparation to prevent such damage thus contributing to the protection against cancer, cardiovascular diseases, respiratory infections, pulmonary diseases, lung infections, atherosclerosis, respiratory diseases, and other age-related diseases caused by cytotoxic, genotoxic, and proinflammatory mechanisms.

TABLE 3
ORAC Values Comparing the Antioxidant Activity of the Vitamin
C Preparation of Example 1 with Known Dietary Antioxidants

| | ORAC | |
|---|---|---|
| Nutrient source | (μM TE/g) | Reference |
| The vitamin C | 1343 | Example 4 |
| preparation of Example 1 | | |
| | trial #1 1062 | |
| | trial #2 1394 | |
| | trial #3 1402 | |
| | trial #4 1440 | |
| Cinnamon | 1243 | Sua et al., 2007 |
| Freeze-Dried Acai | 1027 | Schauss et al., 2006 |
| Green and black teas | 761.1 | Prior and Cao, |
| | (235-1526) | 1999 |
| Chokeberry | 161 | Wu et al., 2004 |
| Broccoli | 65.8 to 121.6 | Kurilich et al., |
| | | 2002 |
| Soft wheat | 32-48 | Moore et al., 2005 |
| Careless gooseberry | 21 | Wu et al., 2004 |

### Example 5

The ability to promote neurite outgrowth was determined for the formulation of Example 1 and other vitamin C formulations.

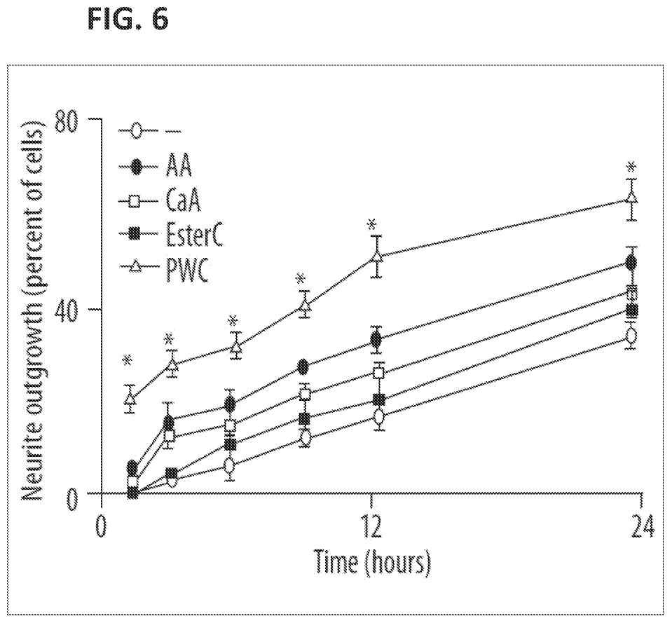

PC12 cells were treated with 100 ng/ml of Nerve Growth Factor (NGF) and incubated for a 24 hour period followed by treatment with either vehicle (−) or various 50 μM of (1) ascorbic acid (AA), (2) calcium ascorbate (CaA), (3) calcium ascorbatecalcium threonate-dehydroascorbate (Ester-C), or (4) the vitamin C preparation of Example 1 (PWC). The formation of neurites was measured at hours 1, 3, 6, 9, 12, and 24. The results are shown in FIG. 6.

PC12 cells responded to NGF treatment by extending neurites. The vitamin C preparation of Example 1 significantly enhanced the NGF-induced neurite outgrowth in 12% of the cells by the first hour. In fact, the vitamin C preparation was the only formulation that resulted in a significant augmentation of NGF-induced neurite outgrowth, suggesting that this is the only formulation that would aid in protection against neurodegenerative diseases. In short, the neurites were twelve-fold more efficient and the superiorly absorbed form of vitamin C promoted nerve regeneration more efficiently than all forms of vitamin C tested at all points in time.

### Example 6

The ability to promote fibroblast adhesion to fibronectin was determined for the formulation of Example 1 and other vitamin C formulations.

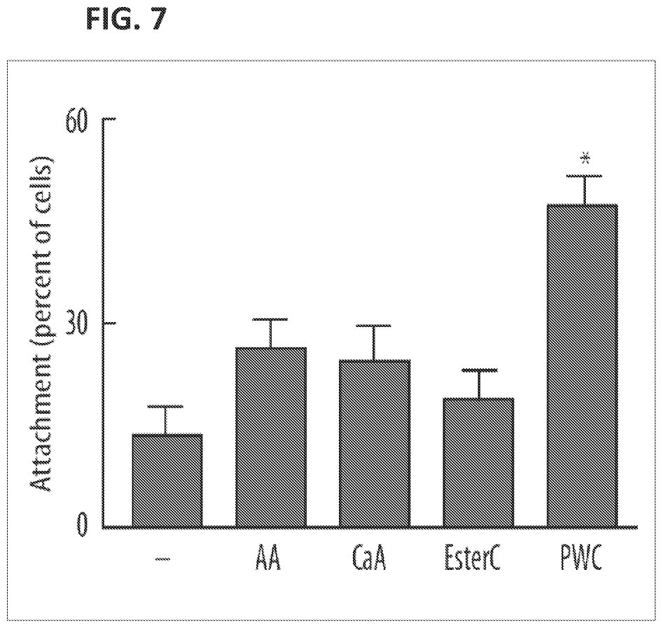

NIH3T3 fibroblastoma cells were seeded onto fibronectin coated plates pretreated with either vehicle (−) or various 50 mM of (1) ascorbic acid (AA), (2) calcium ascorbate (CaA), (3) calcium ascorbate-calcium threonate-dehydroascorbate (Ester-C), or (4) the vitamin C preparation of Example 1 (PWC). The plates were incubated for 15 minutes at 37° C. The unattached cells were removed by aspiration and the attached cells were fixed, stained, and counted in triplicate. Results are shown in FIG. 7.

The vitamin C preparation of Example 1 enhanced fibroblast adhesion to fibronectin by over three-fold. The preparation of Example 1 stimulated wound healing more efficiently than all forms of vitamin C tested at all points in time. In addition to adhesion, fibroblast spreading on fibronectin is an important next step to migration and wound healing performance.

### Example 7

The human serum vitamin C, plasma C-reactive protein, oxidized LDL, and urine uric and oxalate levels were determined for the formulation of Example 1 and other vitamin C formulations.

Healthy volunteers maintained a low vitamin C diet for 14 days. Following an overnight fast, volunteers received a single oral dose of 1000 mg or either (1) ascorbic acid (AA), (2) calcium ascorbate (CaA), (3) calcium ascorbate-calcium threonatedehydroascorbate (commercially available as Ester-C), or (4) the vitamin C preparation of Example 1 (PWC). Blood samples were collected immediately prior to the oral dose administration and at various time points post ingestion. Urine was collected over a 24-hour time period and saved for oxalate and uric acid assays. Serum vitamin C levels were measured by HPLC with coulometric electrochemical detection. Plasma C-reactive protein and oxidized LDL were measured by enzyme linked immunosorbent assay (ELISA) and urine uric acid and oxalate levels were measured by enzymatic methods.

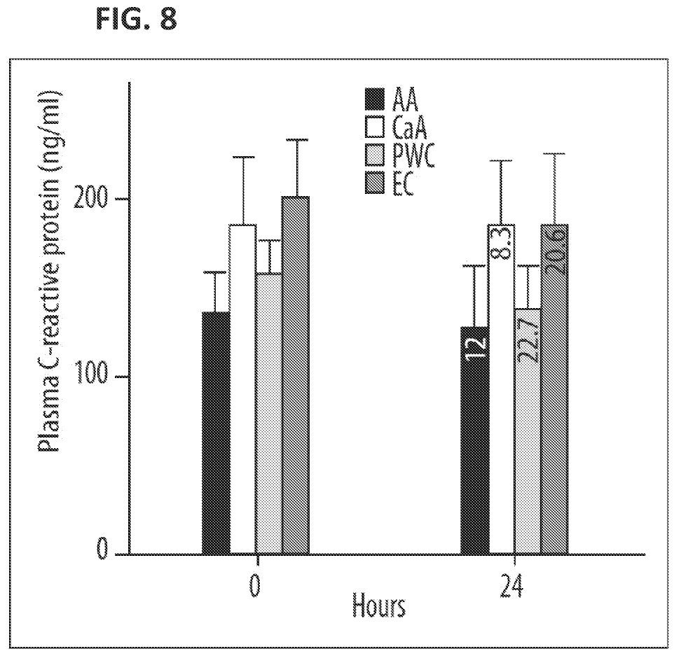

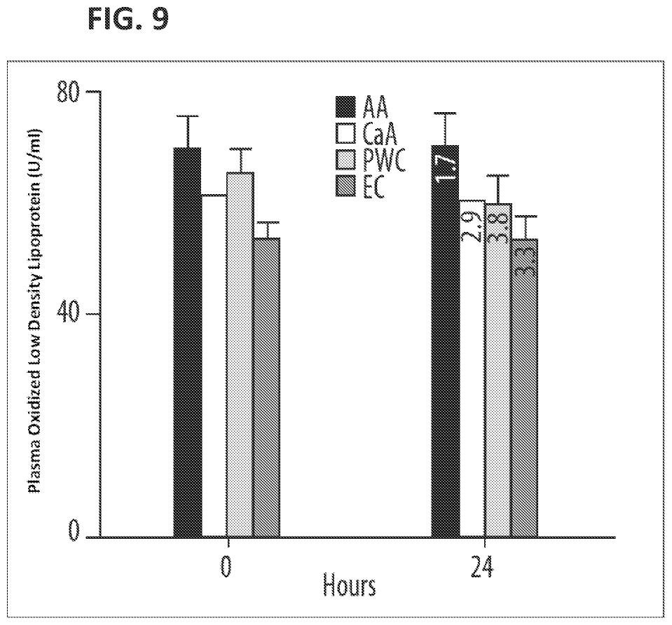

As can be seen in FIG. 8, the preparation of Example 1 best reduces the plasma levels of C-reactive protein (64% higher) and oxidized LDL (83% higher) in human clinical studies. As can be seen in FIG. 9, on the other hand, the preparation of Example 1 delivers effective antioxidant (12% higher) and free radical scavenging activity (11% higher) using the ORAC and DPPH methods.

The vitamin C preparation of Example 1 is more rapidly absorbed and leads to higher serum vitamin C levels and greater reduction of plasma levels of inflammatory and oxidative stress markers than other forms of vitamin C.

TABLE 4
Clinical Data Comparing the Serum Vitamin C Levels, Plasma C-Reactive
Protein, Oxidized LDL Levels, and Urine Uric Acid and Oxalate Levels of the
Vitamin C Preparation of Example 1 with Other Vitamin C Formulations
Serum Vitamin C Levels (mg/dl)
Hrs Post-Admin:

| Vitamin C | 0 | 1 | 2 | 4 | 6 | 24 |
|---|---|---|---|---|---|---|
| AA | 0.56 ± 0.06 | 1.2 ± 0.10 | 1.64 ± 0.18 | 1.51 ± 0.22 | 1.46 ± 0.13 | 0.80 ± 0.09 |
| CaA | 0.50 ± 0.05 | 0.88 ± 0.10 | 1.12 ± 0.17 | 1.03 ± 0.13 | 1.0 ± 0.13 | 0.59 ± 0.09 |
| Ester-C | 0.56 ± 0.09 | 1.3 ± 0.08* | 2.17 ± 0.19* | 1.54 ± 0.14* | 1.51 ± 0.19* | 0.85 ± 0.08 |
| *PWC | 0.60 ± 0.08 | 1.22 ± 0.11* | 1.69 ± 0.27 | 1.52 ± 0.16* | 1.17 ± 0.12 | 0.73 ± 0.07 |

TABLE 4
Clinical Data Comparing the Serum Vitamin C Levels, Plasma C-Reactive Protein,
Oxidized LDL Levels, and Urine Uric Acid and Oxalate Levels of the Vitamin
C Preparation of Example 1 with Other Vitamin C Formulations (Contd.)

| | Plasma C-Reactive Protein | | | Plasma OxLDL | | | Urine Markers | |
|---|---|---|---|---|---|---|---|---|
| | (ng/ml) | | | (U/ml) | | | (mg/dl) | |
| | 0 | 24 | Change | 0 | 24 | Change | Uric Acid | Oxalate |
| AA | 129.75 ± 26 | 117.00 ± 33 | 12.75 | 68.78 ± 6 | 67.89 ± 5 | 0.89 | 50.85 ± 8.8 | 18.8 ± 2.7 |
| CaA | 189.17 ± 41 | 180.83 ± 43 | 8.34 | 60.56 ± 5 | 57.67 ± 6 | 3.78 | 39.75 ± 10.5 | 17.8 ± 2.6 |
| Ester-C | 152.30 ± 19 | 128.60 ± 19 | 23.7 | 62.56 ± 5 | 57.30 ± 4 | 5.26** | 48.73 ± 7.1 | 13.7 ± 1.5 |
| *PWC | 200.63 ± 38 | 180.00 ± 52 | 20.63 | 50.51 ± 4 | 48.20 ± 4 | 2.31 | 40.96 ± 7.0 | 17.9 ± 1.9 |

*Statistically significant deference compared to Calcium Ascorbate. At one hour p = 0.0026 for PWC and p = 0.049 for Ester-C. At two hours, p = 0.0009. At four hours p = 0.0278 for PWC and 0.0477 for Ester C. At six hours, p = 0.0470.
**Statistically significant difference from Ascorbic Acid (p = 0.045). Note that the reductions in oxLDL were not significantly different for any vitamin C supplementation with a before-and-after comparison; however, the drop observed with PWC was significantly greater than the drop observed with Ascorbic Acid.
Note:
All statistically significant differences are noted. Data are presented as the mean + S.E.M. All 0 time points were immediately prior to oral administration of the vitamin C formulation.

### Example 8

The human serum vitamin C, plasma C-reactive protein, oxidized LDL, and urine uric and oxalate levels were determined for the formulation of Example 1 and other vitamin C formulations.

Healthy volunteers maintained a low vitamin C diet for 14 days. Following an overnight fast, volunteers received a single oral dose of 1000 mg or either (1) ascorbic acid (AA), (2) calcium ascorbate (CaA), (3) calcium ascorbate-calcium threonatedehydroascorbate (commercially available as Ester-C), or (4) the vitamin C preparation of Example 1 (PWC). Blood samples were collected immediately prior to the oral dose administration and at various time points post ingestion. Urine was collected over a 24-hour time period and saved for oxalate and uric acid assays. Serum vitamin C levels were measured by HPLC with coulometric electrochemical detection. Plasma C-reactive protein and oxidized LDL were measured by enzyme linked immunosorbent assay (ELISA) and urine uric acid and oxalate levels were measured by enzymatic methods.

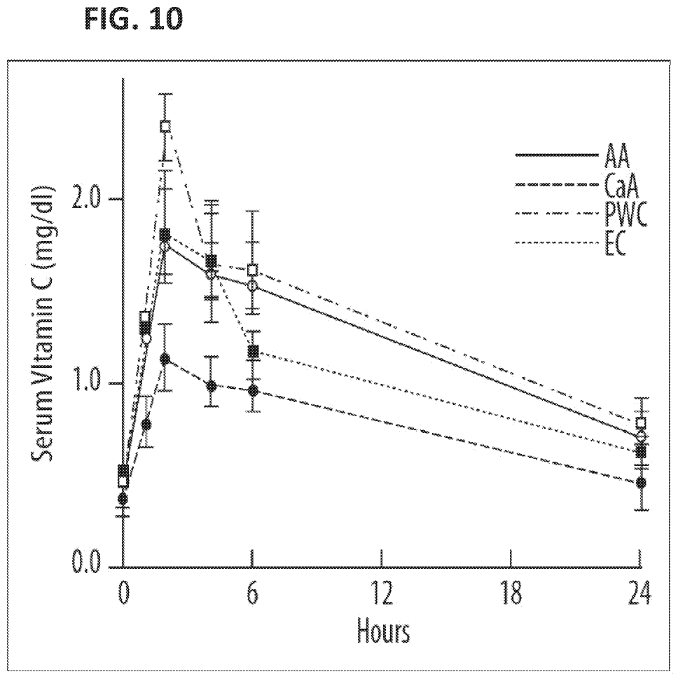

The vitamin C preparation of Example 1 is more rapidly absorbed and leads to higher retained serum vitamin C levels and greater reduction of plasma levels of inflammatory and oxidative stress markers than other forms of vitamin C. Results are shown in FIG. 10.

### REFERENCES

1) Bessey O, Lowry O, Brock M: The quantitative determination of ascorbic acid in small amount of white blood cells and platelets. JBC 1947, 168(1):197-205;
2) Boothby L A, Doering P L: Vitamin C and vitamin E for Alzheimer's disease. Ann. Pharmacotherapy 2005, 39(12):2073-80;
3) Fay M J, Bush M J, Verlangieri A J: Effect of aldonic acids on the uptake of ascorbic acid by 3T3 mouse fibroblasts and human T lymphoma cells. Gen. Pharmacol. 1994, 25(7):1465-69;
4) Kaplan B, Gonul B, Dincer S, Dincer Kaya F N, Babul A: Relationships between tensile strength, ascorbic acid, hydroxyproline, and zinc levels of rabbit full-thickness incision wound healing. Surg. Today 2004, 34(9):747-51;
5) Kurilich A C, Jefferey E H, Juvik J A, Wallig M A, and Klein B P: Antioxidant capacity of different broccoli (Brassica oleracea) genotypes using the oxygen radical absorbance capacity (ORAC) assay. J. Agric. Food Chem. 2002, 50(18):5053-57;
6) Landmark K: Could intake of vitamins C and E inhibit development of Alzheimer dementia? Tidsskr Nor Laegeforen 2006, 15(8):159-61;
7) Lehr H A, Frei B, Arfors K E: Vitamin C prevents cigarette smoke-induced leukocyte aggregation and adhesion to endothelium in vivo. PNAS 1994, 91(16):7688-92;
8) Marionnet C, Vioux-Chagnoleau C, Pierrard C, Sok J, Asselineau D, Bernerd F: Morphogenesis of dermal-epidermal junction in a model of reconstructed skin: beneficial effects of vitamin C. Exp. Dermatol. 2006, 15(8):625-33;
9) Moore J, Hao Z, Zhou K Luther M, Costa J, Y L: Carotenoid, tocopherol, phenolic acid, and antioxidant properties of Maryland-grown soft wheat. J. Agric. Food Chem. 2005, 53(17):6649-57;
10) Pancorbo D, Vazquez C, Fletcher M A: Vitamin C-lipid metabolites: Uptake and retention and effect on plasma C-reactive protein and oxidized LDL levels in healthy volunteers. Med. Sci. Monit. 2008, 14(11):CR547-551;
11) Prior R L, Cao G: Antioxidant capacity and polyphenolic components of teas: implications for altering in vivo antioxidant status. Proc. Soc. Exp. Biol. Med. 1999, 220(4):255-61;
12) Schauss A G, Xianli W., Prior R L, Ou B, Huang D, Owens J, Agarwal A, Jensen G S, Hart A N, Shanbrom E: Antioxidant capacity and other bioactivities of the freeze-dried Amazonian palm berry, Euterpe oleraceae Mart. (Acai). J. Agric Food Chem. 2006, 54(22):8604-10;
13) Su L, Yin J-J, Charles D, Zhou K, Moore J, and Yu L: Total phenolic contents, chelating capacities and radical-scavenging properties of black peppercorn, nutmeg, rosehip, cinnamon and oregano leaf. Food Chem. 2007, 100(3):990-97;
14) Weber C, Erl W, Weber K, Weber P C: Increased adhesiveness of isolcated monocytes to endothelium is prevented by vitamin C intake in smokers. Circulation 1996, 93(8):148892;
15) Weeks B S and Perez P P: A novel vitamin C preparation enhances neurite formation and fibroblast adhesion and reduces xenotiotic-induced T-cellhyperactivation. Med. Sci. Monit. 2007, 13(3):BR51-58;
16) Weeks B S and Perez P P: Absorption rates and free radical scavenging values of vitamin C-lipid metabolites in human lymphoblastic cells. Med. Sci. Monit. 2007, 13(10):BR205-210;
17) Wu X, Gu L, Prior R L, McKay S: Characterization of anthocyanins and proanthocynaidins in some cultivars of Ribes, Aronia, and Sambucus and their antioxidant capacity. J. Agric. Food Chem. 2002, 52(26):7846-56; and
18) Zhou X, Tai A, YamamotoI: Enhancement of neurite outgrowth in PC12 cell stimulated with cyclic AMP and NGF by 6-acylated ascorbic acid 2-O-alpha-glucosides (6-AcylAA-2G), novel lipophilic ascorbate derivatives. Biol. Pharm. Bull. 2003, 26(3):341-46.

Since many modifications, variations and changes in detail can be made to the described embodiments of the invention, it is intended that all matters in the foregoing description and shown in the accompanying drawings be interpreted as illustrative and not in a limiting sense. Thus, the scope of the invention should be determined by the appended claims and their legal equivalents.

What is claimed is:

1. A method for improving the status of a health condition comprising:
identifying a health condition, and
administering a vitamin C preparation comprising a vitamin C component, the vitamin C component comprising at least about 90% by weight of the total weight of the vitamin C preparation.

2. The method as recited in claim 1, wherein the health condition comprises nervous system health.

3. The method as recited in claim 1, wherein the health condition comprises a risk of developing a neurodegenerative disease.

4. The method as recited in claim 1, wherein the health condition comprises neurite outgrowth via NGF mediation.

5. The method as recited in claim 1, wherein the health condition comprises wound healing.

6. The method as recited in claim 1, wherein the health condition comprises fibroblast adhesion to and the interaction with human extracellular matrices.

7. The method as recited in claim 1, wherein the health condition comprises a protection offered by human immune systems against xenobiotics.

8. The method as recited in claim 1, wherein the health condition comprises a risk of developing an oxidative pathogenesis.

9. The method as recited in claim 1, wherein the health condition comprises a risk of developing cancer.

10. The method as recited in claim 1, wherein the health condition comprises a risk of developing cardiovascular diseases.

11. The method as recited in claim 1, wherein the health condition comprises a risk of developing respiratory infections.

12. The method as recited in claim 1, wherein the health condition comprises a risk of developing pulmonary diseases.

13. The method as recited in claim 1 wherein the health condition comprises a risk of developing lung infections.

14. The method as recited in claim 1, wherein the health condition comprises a risk of developing atherosclerosis.

15. The method as recited in claim 1, wherein the health condition comprises a risk of developing respiratory diseases.

16. The method as recited in claim 1, wherein the health condition comprises a risk of developing age-related diseases associated with cytotoxic mechanisms.

17. The method as recited in claim 1, wherein the health condition comprises a risk of developing age-related diseases associated with genotoxic mechanisms.

18. The method as recited in claim 1, wherein the health condition comprises a risk of developing age-related diseases associated with proinflammatory mechanisms.

19. The method as recited in claim 1, wherein the vitamin C formulation further comprises a lipophilic molecule component, said lipophilic molecule component comprising lipophilic molecules at least about 0.1% by weight of the total weight of the vitamin C preparation.

20. The method as recited in claim 19, wherein the lipophilic molecule component further comprises at least one saturated straight C₁₆-C₃₄ fatty alcohol and at least one unsaturated ω-9 C₁₈-C₂₄ fatty acid.
